(19) United States
(12) Patent Application Publication
(10) Pub. No.: US 2017/0136123 A1
(43) Pub. Date: May 18, 2017

(54) STANNOUS DOPED MICRO AND NANO PARTICLES FOR AUGMENTED RADIOFREQUENCY ABLATION

(71) Applicant: Amrita Vishwa Vidyapeetham, Kochi (IN)

(72) Inventors: MANZOOR KOYAKUTTY, Kochi (IN); VIJAY HARISH, Kochi (IN); SHANTIKUMAR NAIR, Kochi (IN)

(73) Assignee: Amrita Vishwa Vidyapeetham, Kochi (IN)

(21) Appl. No.: 15/314,468

(22) PCT Filed: Jan. 10, 2014
(86) PCT No.: PCT/US14/11153

§ 371 Date: Nov. 28, 2016

(30) Foreign Application Priority Data
Jan. 10, 2013 (IN) 156/CHE/2013

(51) Int. Cl. A61K 41/00; A61B 18/18; A61K 31/704; A61K 9/50; A61K 9/51

(52) U.S. Cl. CPC A61K 41/0052; A61K 9/5015; A61K 9/5115; A61K 9/5161; A61K 9/5031; A61K 47/48923; A61K 31/704; A61K 47/48907; A61K 9/5169; A61K 47/48915; A61K 9/5153; A61B 18/18; A61B 2018/00577

(57) ABSTRACT

A composition for facilitating localized delivery of energy and of therapeutic agents to a lesion in tissue is disclosed comprising nanoparticles of a polysaccharide, gelatin or a polymer, wherein the nanoparticles are complexed with tin ions. The composition may comprise one or more therapeutic agents. The composition may be configured to improve the thermal effect of RF energy on a lesion and or to release the therapeutic agents to the lesion when RF energy is applied.

### CROSS-REFERENCES TO RELATED APPLICATIONS

This application claims the benefit and priority of the following: Indian Patent Application No. 156/CHE/2013, filed on Jan. 10, 2013, the full disclosure of which is incorporated herein by reference.

### FIELD OF THE INVENTION

This disclosure relates generally to treatment of lesions and particularly to a method of localized ablation of lesions using stannous-doped nanoparticles.

### DESCRIPTION OF THE RELATED ART

Radiofrequency ablation (RFA) is an established treatment technique being practiced clinically. It is based on the principle of hyperthermia for the destruction of diseased tissues. Known problems with conventional ablation techniques involve non-uniform heating of tissue, non-targeted heating of both diseased and healthy tissue, excessive heating causing desiccation or charring of the tissue etc. The major limitation of the conventional RFA method is small size of the lesion that can be effectively treated. This issue is dealt with by repeatedly repositioning the radiofrequency (RF) electrodes to cover a larger area but this requires very precise movement and patient compliance. Attempts have been made to provide an RF device that increases the lesion size by increasing electrode size or using multipronged electrodes. While such devices have produced some increment in the size of the ablated region, larger treatment volumes are still desired.

There is also a need in tumor treatment for a formulation that can augment the efficacy of RFA while simultaneously providing an embolization effect so as to block the blood flow to the region and thus reduce the heat sink effect due to blood flowing through the region and ultimately improve the therapeutic outcome.

Alteration of radiofrequency ablation using normal saline and other salt solutions has been evaluated in an attempt to more efficiently destroy diseased tissue, especially solid tumors. U.S. Pat. No. 7,510,555 discloses use of aqueous solutions of metal sulfates or any injectable salt solutions as RF absorption enhancers.

Targeting of RF enhancing nanoparticles or nanoconstructs have also been described which allow target-specific accumulation of the nanoparticles and hence a more localized ablation.

This application discloses compositions for ablation and/or delivery of therapeutic agents using nanoparticles complexed with metal ions that address some of the drawbacks discussed above.

### SUMMARY OF THE INVENTION

A composition for facilitating localized delivery of energy and therapeutic agents to a lesion in tissue is disclosed, that comprise nanoparticles. The nanoparticles comprise at least one of a polysaccharide, gelatin or a polymer, and are complexed with tin ions. The composition may further comprise one or more therapeutic agents. The nanoparticles of the composition may further be coated with galactose. The composition may further be configured to improve the thermal effect of RF energy on a lesion. The composition may also be configured to release the at least one or more therapeutic agents to the lesion when RF energy is applied to the lesion.

The nanoparticles comprising the composition may be sized between 1 nanometer and 1000 nanometers or between 100-200 nanometers. The complexing with tin ions may be accomplished using a stannous precursor in the concentration range 0.001 micromolar to 1 molar.

A method of treating a lesion is disclosed comprising, delivering nanoparticles comprising at least one of a polysaccharide, gelatin or a polymer to a target lesion, wherein the nanoparticles are complexed with tin ions, and delivering RF energy to the target lesion. The nanoparticles used may be coupled to a therapeutic agent and the method may further comprise releasing the therapeutic agent when the RF energy is delivered to the target lesion. The method may also comprise modulating the release of the therapeutic agent by controlling the delivery of the RF energy to the target lesion.

A method of formulating a nanoparticle composition is disclosed comprising, complexing nanoparticles including at least one of a polysaccharide, gelatin or a polymer with tin ions. The method of formulating a nanoparticle composition may further comprise coupling one or more therapeutic agents to the nanoparticles.

### BRIEF DESCRIPTION OF THE DRAWINGS

Present embodiments have other advantages and features which will be more readily apparent from the following detailed description and the appended claims, when taken in conjunction with the accompanying drawings, in which:

FIG. 1 illustrates a method of obtaining compositions including tin-doped nanoparticles for RF ablation and delivery of therapeutic agents to tissue.

FIG. 2 shows a process of producing galactosylated tin-doped nanoparticles for RF ablation and delivery of therapeutic agents.

FIG. 3 illustrates a mechanism of galactosylation of tin-doped nanoparticles.

FIG. 4A shows particle size of stannous doped alginate nanoparticles by DLS analysis.

FIG. 4B is an atomic force microscopic image of the stannous doped alginate nanoparticles showing particle size distribution between 70 nm to 100 nm.

FIG. 4C shows DLS and AFM analysis (inset) of the same particles after galactosylation showing an increase in size range to 100-200 nm.

FIG. 5 shows FTIR spectrum of stannous doped nanoparticles: alginate stannous nanoparticles, alginate with PEI coating and the galactosylated nanoparticle with PEI coating.

FIG. 6 shows heating response of Sn-alginate nanoparticles compared with Sn salt or alginate solution.

FIG. 7 shows release kinetics of doxorubicin co-loaded compositions, A) without RF ablation and B) with RF ablation.

FIG. 8 illustrates the relative toxicity to HEPG2 cell line seen on exposure to plain doxorubicin drug, showing superior performance of galactosylated stannous doped compositions containing doxorubicin with RF ablation.

FIG. 9A and 9B show biodistribution of stannous doped nanoparticle compositions in Wistar rat: A) plain stannous doped composition and B) galactosylated stannous doped composition.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

The present disclosure relates to stannous doped micro- and nano-sized polymeric/protein or polysaccharide compositions that produce heat energy under an alternating radio-frequency current or field. These compositions can be used for the embolization of the feeding blood vessels, local delivery of therapeutic agents and simultaneously producing targeted thermal ablation of malignant as well as benign lesions in the body. In various embodiments the compositions comprise tin (including stannous), doped polymer, protein, oligosaccharide or polysaccharide materials of size varying from nano to micrometers. In one aspect this present disclosure provides a method for formulating tin doped nano/micro particles of varying sizes that can cause local hyperthermia response under the influence of radio-frequency waves (100 Hz-100 GHz), either invasively or non-invasively. In an embodiment, the process of producing such a formulation consists of providing precursors for preparing the stannous doped polymers/proteins or polysaccharides and preparing nano and/or micron sized stannous doped polymers/proteins or polysaccharides of size varying from 1-1000 nanometers and/or 1-1000 micrometers.

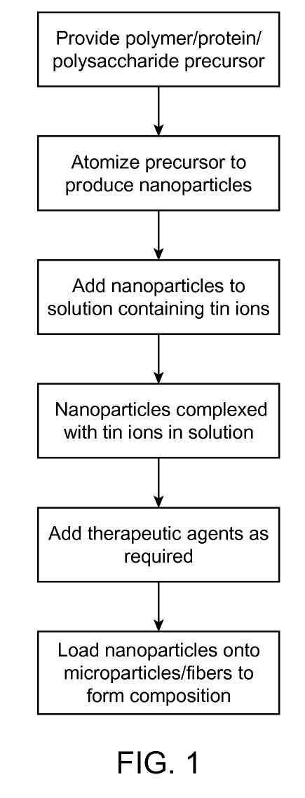

In one embodiment, said constructs are prepared by doping, complexing or mixing of stannous. A composition for facilitating localized delivery of energy and therapeutic agents to a lesion in tissue is illustrated in FIG. 1. In step 101, a precursor comprising polymers/proteins or polysaccharides is taken and atomized (step 102) to form nanodroplets or nanoparticles of the corresponding substance. In some embodiments, the nanodroplets are atomized into a solution containing tin ions in step 103. The nanoparticles are complexed with the tin ions in solution upon mixing the solution for a period of time (step 104). In one embodiment the nanoparticles of the invention is loaded with one or more therapeutic agents (step 105). The nanoparticles of polymer/protein/polysaccharide are then loaded onto suitable vehicle for delivery as a therapeutic composition (step 106).

In various embodiments, the polymer/protein or polysaccharide precursor is selected from one or more of polyvinyl alcohols, polyacrylic acids, polymethacrylic acids, polyethylenimines, poly vinyl sulfonates, alginates, galactomannans, carboxymethyl celluloses, hydroxyethy celluloses, substituted celluloses, polyanhydrides, poly (ortho)esters, polyacrylamides, polyethelene glycols, polyamides, polyvinylpyrrolidons, polyureas, polyurethanes, polyesters, polyethers, polustyrenes, other polysaccharides (like chitin, chitosan, agar, carboxymethy chitin), polylactic acids, polyethylenes, polymethylmethacrylates, polycaprolactones, polyvinyl acetate, polyglycolic acids, poly(lactic-co-glycolic) acids, proteins such as gelatin or collagen, monomers such as mannose, sucrose, starch, or oligomers and their different combinations thereof.

In some embodiments the nanoparticles may varying in size from 1-1000 nanometers. In some embodiments the nanoparticles may have an average diameter of about 100-200 nanometers.

In various embodiments the tin used for complexing with the nanoparticles is of oxidation state (II) or (IV). In some embodiments the tin precursor is at least one of stannous chloride, stannous carbonate, stannous phosphate stannous fluoride or other inorganic and organic complexes of tin. In some embodiments the concentration of the tin precursor varies from 0.001 micromolar to 1 molar.

The therapeutic agents may comprise alkylating agents, antimetabolites, antiangiogenic agents, vinca alkaloids, taxanes, epipodophyllo toxins, antibiotics, camptothecin analogues or curcumin co-loaded or mixed with the stannous doped nanoparticles.

In one aspect of the invention, the nanoparticles are further incorporated into micro spheres or micro fibers produced from the same or different polymers/proteins/polysaccharides to form a composition effective for localized thermal treatment and delivery of therapeutic agents.

In one embodiment, the compositions are optimized to produce a localized thermal effect on RF exposure. In one embodiment the nanoparticles and the microspheres or microfibers are optimized for controlled release of the therapeutic agent.

In one aspect, a method of treating a lesion comprises delivering a composition comprising nanoparticles to a target lesion and delivering RF energy to the lesion. In some embodiments the nanoparticles may comprise a polysaccharide, gelatin or a polymer, and are complexed with tin ions. In some embodiments the nanoparticles are coupled or loaded with a therapeutic agent. In one embodiment the method comprises releasing the therapeutic agent through delivery of RF energy to the target lesion. In one embodiment the method further comprises modulating the release of the therapeutic agent by controlling the delivery of RF energy to the target lesion.

In one embodiment of the method of treatment, the tin-doped nanoparticle compositions are administered by suspending them in a carrier including one or more of normal saline, dextrose normal saline, dextrose solution, Lipiodol, a CT/MRI contrast agent, a polymeric gel and/or sterile water for injection.

In one embodiment of a method of treatment, the tin-doped nanoparticle compositions are instilled intra-arterially. In some embodiments, the method can include disposing a plurality of the nanoparticles into the tissue of interest in a subject by percutaneous injection, intravenous injection, intralesional injection, perilesional injection, subcutaneous injection, intradermal injection, or intracavitary instillation.

In one aspect of a method of treatment, the tin-doped nanoparticles are surface conjugated or blended with, at least one, specific tissue targeting ligand such as a folic acid receptor, mannose receptor, galactose receptor, asialoglycoprotein receptor, antibodies against endothelial growth factor receptor, vascular growth factor receptor, prominin-1 (CD133), CD44, CD123, CD24, CD117, CD33, C-kit receptor (Cd117), transferrin receptor, integrin, Her2 receptor, somatostatine receptor, oestrogen receptor, progesterone receptor, prostate specific antigen receptor, mucine protein, p-glycoprotein, mannose, galactose, galactomannans, oligosaccharides, etc. In one embodiment the nanoparticles are coated with galactose for providing affinity to the asialoglycoprotein receptors which are abundantly expressed in the liver (hepatocytes).

In one embodiment of a method of treatment, the nano/micro particle-infused tissue is exposed to a radiofrequency electromagnetic field (100 Hz-100 GHz). The radiofrequency exposure may be provided either non-invasively or invasively using a probe inserted at the site of interest, either percutaneously or intraoperatively.

A method of formulating a composition comprising nanoparticles of polysaccharide, gelatin or a polymer complexed with tin ions is provided. In one aspect the nanoparticles are coupled to one or more therapeutic agents. In one aspect, the nanoparticle compositions of the invention are loaded with therapeutic agents including one or more of alkylating agents, antimetabolites, antiangiogenic agents, vinca alkaloids, taxanes, epipodophyllo toxins, antibiotics, camptothecin analogues and curcumin. In some embodiments the effects of different treatment techniques are combined, for improved treatment effects.

### EXAMPLES

### Example 1

### Stannous Doped Alginate Nanoparticles with PEI Coating

In this example, preparation of stannous-doped alginate nanoparticles is presented. Stock solutions of sodium alginate 0.8 wt % and of stannous chloride (6 mg/ml) were prepared using distilled water. 4 m1 of the alginate stock was taken in a 10 ml beaker and the stannous chloride solution was aerosprayed into it at a rate of 0.5 ml/hr for 2 hours, with a constant stirring at 1000 rpm. Then, 1 ml of 0.003% polyethyleneimine (PEI) was added to the resulting mixture and stirring maintained at 600 rpm for another 30 minutes. The PEI coated nanoconstructs were recovered from this liquid by centrifugation at 5000 rpm for 5 minutes at 21° C.

### Example 2

### Stannous Doped Doxorubicin Co-Loaded Alginate Nanoconstructs with Galactosylation

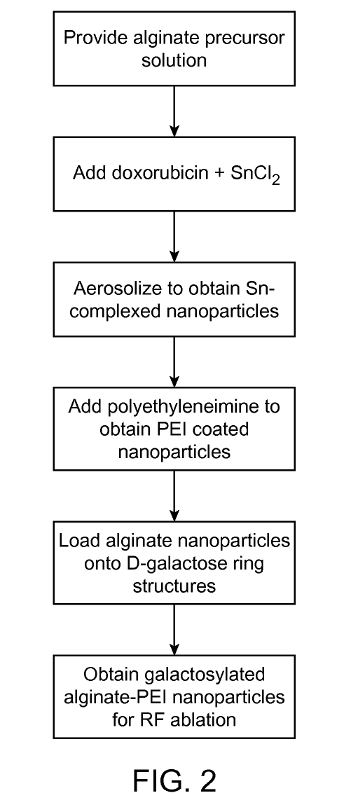

In this example, preparation of a stannous doped alginate nanoconstruct loaded with the chemotherapeutic drug doxorubicin is presented as shown in FIG. 2. In step 201 stock solution of sodium alginate 0.8 wt % was taken. Doxorubicin and stannous chloride each of 3 mg/ml concentration were prepared using distilled water (step 202). In step 203, 8 ml of the alginate stock was taken in a 10 ml beaker and the doxorubicin-stannous chloride solution aerosprayed into it at a rate of 0.5 ml/hr for 2 hours, with a constant stirring at 1000 rpm, to obtain Sn-complexed nanoparticles comprising doxorubicin suspended in the liquid. Then, in step 204 1 ml of 0.06% polyethyleneimine (PEI) was added to the resulting mixture and stirring maintained at 600 rpm for another 30 minutes. The PEI coated nanoconstructs were recovered by centrifugation at 5000 rpm for 5 minutes at 21° C. In step 205, for the galactosylation process, first 1 μM of D-galactose was slowly added drop wise to PEI treated alginate doxorubicin nanoparticles and incubated for 15 minutes at room temperature. The resultant solution mixture was centrifuged at 5000 rpm for 5 minutes at 21° C. and washed thoroughly in distilled water to remove any un-reacted galactose or partially galactosylated nanoparticles. Thus, the resulting product in step 206 comprised galactosylated PEI coated nanoparticles complexed with tin and loaded with doxorubicin.

### Example 3

### Galactosylation of Alginate Nanoparticles

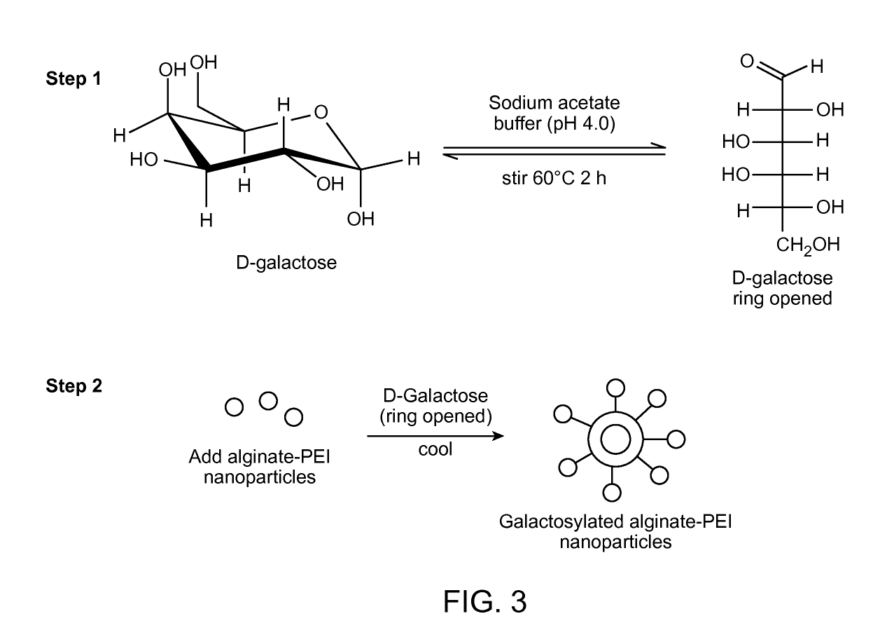

The galactosylation of alginate nanoparticles using the methods of the invention is illustrated as shown in FIG. 3. D-galactose in cyclic form was taken in solution and stirred at 60° C. for 2 hours in presence of sodium acetate buffer at a pH of 4.0 to open the ring structure in step 1. In step 2, the PEI coated alginate nanoparticles as shown in FIG. 2 were added to this solution and upon cooling, galactose encapsulated PEI coated stannous-doped alginate nanoparticles were obtained.

### Example 4

### Characterization of Stannous-Doped Alginate Nanoparticle Compositions

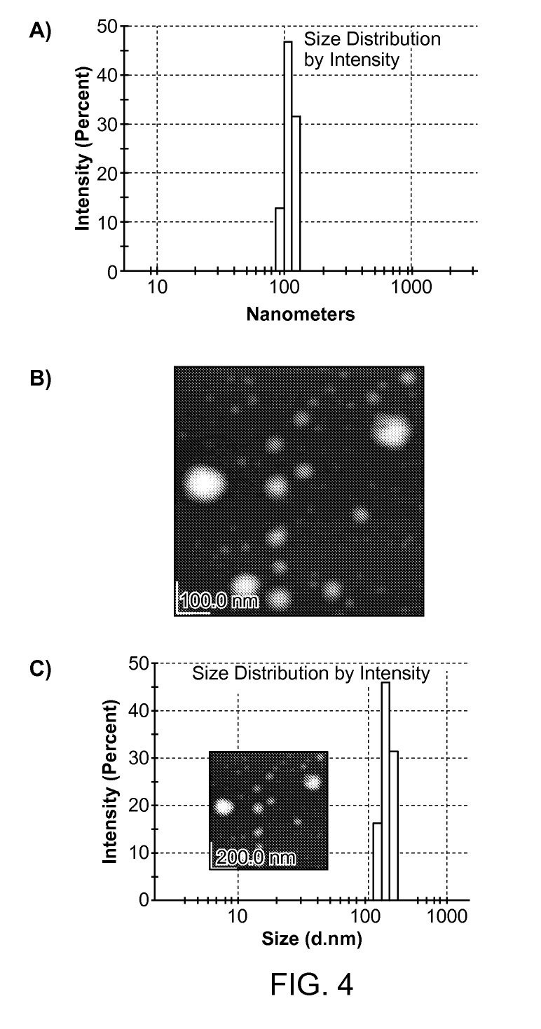

The stannous doped alginate nanoparticle compositions prepared as illustrated in Examples 2 and 3 were characterized using DLS and AFM analysis. Particle size of the stannous-doped alginate nanoparticles measured by DLS was in the range 70-100 nm as shown in FIG. 4A. The range of sizes of the particles was confirmed by atomic force microscopy imaging (FIG. 4B). The particle sizing by DLS and AFM after galactosylation is shown in FIG. 4C, where the size has increased to 100-200 nm.

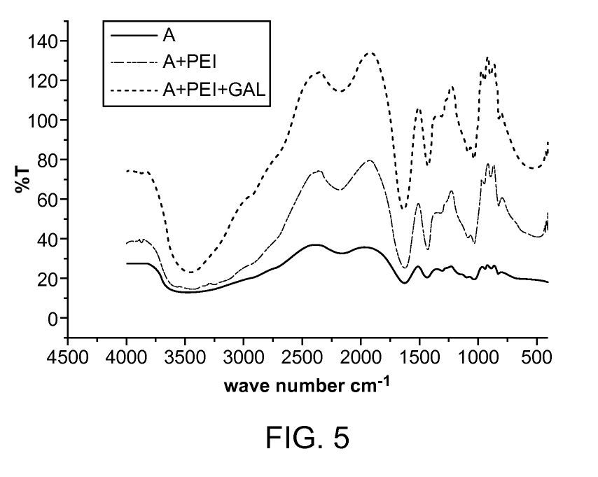

Fourier transform infrared (FTIR) spectroscopic characterization of the nanoparticle compositions of the invention in three conditions is illustrated in FIG. 5. The three curves in FIG. 5 show FTIR spectra of stannous doped alginate nanoparticles, the same particles after PEI coating and the PEI coated composition after galactosylation.

### Example 4

### RF Heating Response and Drug Release Characteristics

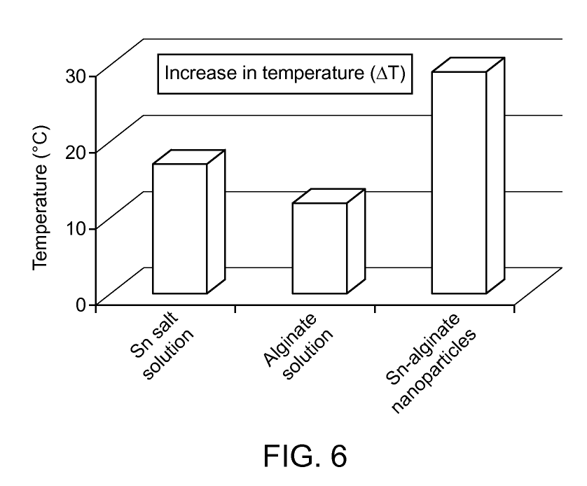

Heating response of a solution comprising the stannous-doped alginate in Example 1 compared to Sn salt solution and plain alginate solution is illustrated in FIG. 6. The solutions were exposed to a uniform RF field of 13.56 MHz, at 100 Watt power for 1 minute. The increase in temperature for stannous and alginate solutions were 17° and 12° C. respectively, while the solution comprising the inventive nanoparticle composition made using the same concentrations of tin and alginate (as solution) showed a temperature increase of 29° C., illustrating the strong coupling of the Sn-doped nanoparticles with the RF for increased heating.

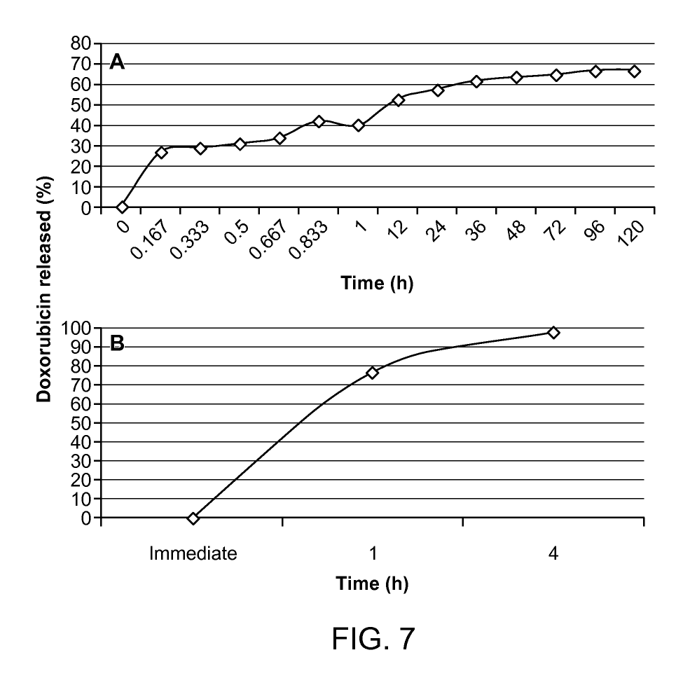

Release characteristics of doxorubicin from the co-loaded stannous doped nanoparticle composition in Example 2 with and without exposure to RF field were studied as a function of time. Without RF exposure, there was a gradual release of the drug which plateaus at 24 hours, by which time around 60% of the drug was released, as shown in FIG. 7A. On exposure to a uniform RF field for 1 minute, rate of drug release increased exponentially (FIG. 7B) with more than 70% of the drug released within 1 h of exposure and nearly 100% release occurring by 4 hours.

### Example 5

### Stannous Doped Gelatin Microparticles

In this example, preparation of stannous doped gelatin microparticles is discussed. Stock solutions of acetone, 0.5 wt % Tween 80 and 0.5 wt % span 80 were placed in cold-room to bring the temperature to 4° C. 250 ml of olive oil was taken in a round bottom flask, to which 250 ml of Tween 80 was added. Then 1.25 ml of span 80 was added under stirring at 250 rpm. 5 g of gelatin dissolved in 45 ml of distilled water at 60° C., ensuring it did not boil over. The gelatin solution was added drop by drop to the initial reaction mixture with stirring at 500 rpm. The flask was then kept in ice-bath for 30 minutes. 100 ml of cold acetone was added. Mixture of 0.5 wt % stannous chloride and 0.1 wt % Tween 80 was added to the reaction mixture. Stannous complexed gelatin nanoparticles in the resulting solution were extracted by filtration and lyophilisation.

### Example 6

### Stannous Doped PLGA Constructs

5 mg stannous chloride was dissolved in 0.75 mL 0.01 M hydrochloric acid (HCl) solution. 0.75 mL of this solution was mixed with 0.75 mL 10 mM phosphate buffer (pH 7.8) to form a nano-suspension. This suspension was poured into 10 mL of dichloromethane (DCM) containing 300 mg PLGA and sonicated in an ice bath for 2 min at output level 10 (50 W) for 2 min to form a W/O emulsion. This primary emulsion was added to 100 mL of 1% PVA solution and sonicated in an ice bath at output level 10 for 2 min to form a W/O/W multiple emulsion in a 250 mL beaker. This multiple emulsion was stirred at room temperature by a magnetic stirrer for 3 h to evaporate the organic solvent and extract the stannous doped nanoparticle composition.

### Example 7

### Efficacy of Interventions Using Compositions of the Invention

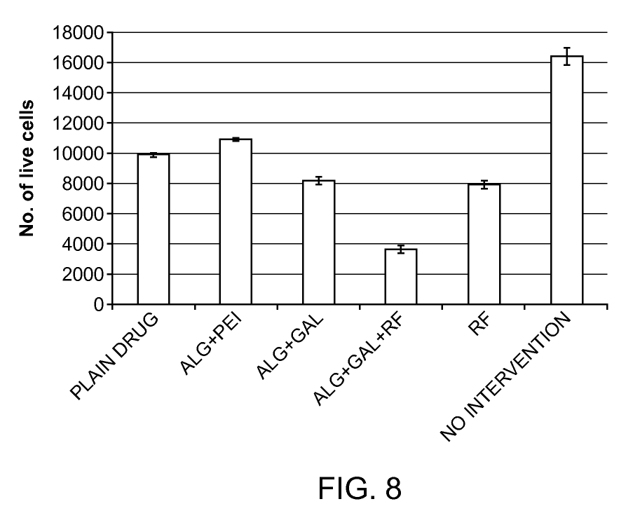

Relative toxicity of various treatments including doxorubicin injection, injection of PEI coated alginate, galactosylated alginate, galactosylated PEI coated alginate with RF ablation and plain RF ablation to HEPG2 cell line was investigated in vitro. The bar-graph representing the relative toxicity to HEPG2 cell line on exposure to the above conditions is shown in FIG. 8. The high cellular toxicity of stannous doped PEI coated alginate nanoparticles used with RF ablation towards HEPG2 is evident from the figure.

### Example 8

### Biodistribution Studies Using Compositions of the Invention

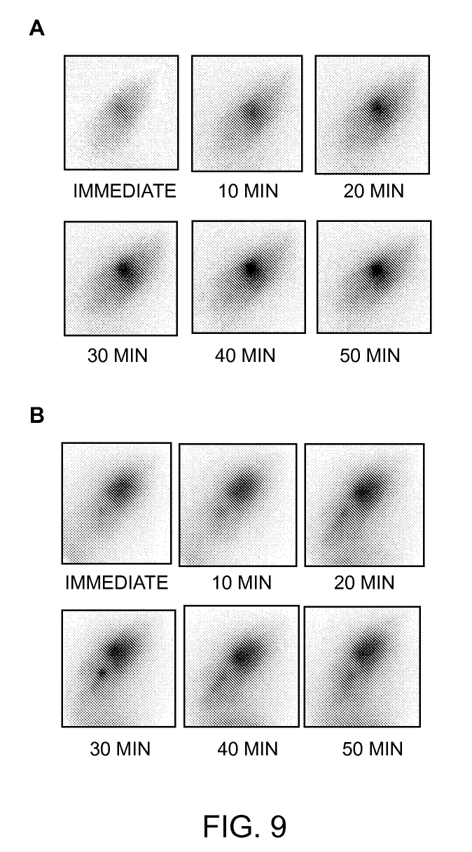

Biodistribution studies were conducted in Wistar rat to demonstrate the efficacy of galactosylation in improving tissue penetration and bioavailability of the inventive compositions. Stannous doped alginate nanoparticles as prepared in Example 1 and 2 were radiolabelled with 99 mTc to allow in-vivo imaging. The distribution of the nanoparticles in Wistar rat liver was studied using radioimaging as shown in FIG. 9. As shown in FIG. 9A non-galactosylated or plain nanoparticles show peak accumulation in the liver only by 40 minutes of exposure, whereas, galactosylated nanoparticle compositions showed much more rapid accumulation in liver with peak activity seen at 20 minutes, as shown in FIG. 9B.

While the above has been disclosed with reference to certain embodiments, it will be understood by those skilled in the art that various changes may be made and equivalents may be substituted without departing from the scope of the invention. In addition, many modifications may be made to adapt to a particular situation or material the teachings of the invention without departing from its scope. The above description should not be taken as limiting the scope of the invention which is defined by the appended claims.

What is claimed is:

1. A composition for facilitating localized delivery of energy and therapeutic agents to a lesion in tissue comprising:
nanoparticles comprising at least one of a polysaccharide, gelatin or a polymer, wherein the nanoparticles are complexed with tin ions.

2. The composition of claim 1, further comprising one or more therapeutic agents. The composition of claim 1, wherein the nanoparticles are coated with galactose.

4. The composition of claim 1, wherein the composition is configured to improve the thermal effect of RF energy on a lesion.

5. The composition of claim 4, further comprising one or more therapeutic agents, wherein the composition is configured to release the at least one or more therapeutic agents to the lesion when RF energy is applied to the lesion.

6. The composition of claim 1, wherein the nanoparticles are sized between 1 nanometer and 1000 nanometers.

7. The composition of claim 1, wherein the nanoparticles are sized between 100-200 nanometers.

8. The composition of claim 1, wherein the complexing with tin ions is accomplished using a stannous precursor in the concentration range 0.001 micromolar to 1 molar.

9. A method of treating a lesion comprising:
delivering nanoparticles comprising at least one of a polysaccharide, gelatin or a polymer to a target lesion, wherein the nanoparticles are complexed with tin ions; and
delivering RF energy to the target lesion.

10. The method of claim 9, wherein the nanoparticles are coupled to a therapeutic agent.

11. The method of claim 10 further comprising releasing the therapeutic agent when the RF energy is delivered to the target lesion.

12. The method of claim 11 further comprising modulating the release of the therapeutic agent by controlling the delivery of the RF energy to the target lesion.

13. A method of formulating a nanoparticle composition comprising:
complexing nanoparticles comprising at least one of a polysaccharide, gelatin or a polymer with tin ions.

14. The method of formulating a nanoparticle composition of claim 13, further comprising coupling one or more therapeutic agents to the nanoparticles.
